(19) United States
(12) Patent Application Publication
(10) Pub. No.: US 2026/0124345 A1
(43) Pub. Date: May 7, 2026

(54) Blood Processing Systems

(71) Applicant: Fenwal, Inc., Lake Zurich, IL (US)

(72) Inventors: Benjamin E. Kusters, Pleasant Prairie, WI (US); Kyungyoon Min, Kildeer, IL (US)

(21) Appl. No.: 19/373,304

(22) Filed: Oct. 29, 2025

Related U.S. Application Data
(60) Provisional application No. 63/716,159, filed on Nov. 4, 2024.

(51) Int. Cl. A61M 1/02; B04B 5/04

(52) U.S. Cl. CPC A61M 1/0245; A61M 1/0218; B04B 5/0442; A61M 2202/0439

(57) ABSTRACT

A blood processing system includes a reuseable processing device and a single use fluid flow circuit. The reuseable hardware component includes a pump system, a valve system, and a centrifuge. The reuseable hardware component further includes a controller and holders for collection containers. The disposable fluid flow circuit includes a plurality of collection containers, a processing chamber received by the centrifuge and a plurality of conduits fluidly connecting the components of the fluid flow circuit. The controller is configured to command the pump system and the valve system to cooperate to convey whole blood from a blood source to the processing chamber and execute at least one blood processing procedure. The holders are configured so that the collection containers remain upright during the duration of the at least one blood processing procedure.

### CROSS-REFERENCE TO RELATED APPLICATIONS

This application claims the benefit of and priority to U.S. Provisional Patent Application Ser. No. 63/716,159, filed Nov. 4, 2024, the disclosure of which is incorporated herein by reference.

### FIELD OF THE DISCLOSURE

This application relates generally to blood and blood component separating, treating and/or processing, and related apparatus, systems and methods associated with such separating and/or processing.

### DESCRIPTION OF RELATED ART

It is well known to collect whole blood from donors using manual collection procedures through blood drives, donor visits to blood centers or hospitals and the like. In such procedures, blood is typically collected by simply flowing it from the donor under the force of gravity and venous pressure into a collection container (e.g., a flexible pouch or bag). Various blood collection instruments may also be used to aid or expedite the collection of blood or blood components.

After initial collection, it is a common practice to transport the collected whole blood to another facility or location, sometimes called a “back lab,” for further processing to form red blood cell (RBC), platelet (PLT) and plasma products from the whole blood, as well as carry out subsequent processes such as cell washing and plasma cryoprecipitate production and collection. This processing usually entails manually loading the primary collection container and associated tubing and satellite containers into a centrifuge to separate the whole blood into concentrated red cells, buffy coat, and platelet-rich or platelet-poor plasma. The separated components may then be expressed from the primary collection container into one or more of the satellite containers and the red cells combined with an additive or preservative solution pre-filled in one of the satellite containers. After the above steps, the blood components may be again centrifuged, if desired, for example to separate platelets from plasma. The overall process requires multiple large floor centrifuges and fluid expression devices. Because of the multiple operator interactions, the process is labor intensive, time consuming and subject to human error.

Thus, there have been continuing efforts to automate the apparatus and systems used in the post-collection processing of whole blood, and recently it has been proposed to employ an automated blood component separator for such post-collection processing. The subject matter disclosed herein provides further advances in various aspects of the apparatus, systems, and methods that may be employed in post-collection processing systems (while also being applicable to processing of blood being drawn from a living donor) by offering devices and system configured to maintain an upright position for the product containers with a particularly configured holder.

### SUMMARY

There are several aspects of the present subject matter which may be embodied separately or together in the devices, systems, and methods described and/or claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto or later amended. For purposes of this description and claims, blood components include concentrated red cells, plasma, platelets and white cells, whether with or without anticoagulant or additives.

By way of the present disclosure, a blood processing system is provided comprising a durable hardware component and a single use fluid flow circuit. The reuseable hardware component includes a pump system, a valve system, and a centrifuge. The reuseable hardware component further includes a controller and holders for collection containers. The disposable fluid flow circuit includes a plurality of collection containers, a processing chamber received by the centrifuge and a plurality of conduits fluidly connecting the components of the fluid flow circuit. The controller is configured to command the pump system and the valve system to cooperate to convey whole blood from a blood source to the processing chamber and execute at least one blood processing procedure. The holders are configured so that the collection containers remain upright during the duration of the at least one blood processing procedure.

These and other aspects of the present subject matter are set forth in the following detailed description of the accompanying drawings.

### BRIEF DESCRIPTION OF THE DRAWINGS

FIG. 1 is a perspective view of an exemplary reusable hardware component of a blood processing system which is configured to receive a disposable fluid flow circuit;

FIG. 2 is a plan view of an exemplary disposable fluid flow circuit for use in combination with the durable hardware component of FIG. 1;

FIG. 3 is a schematic view of a biological fluid processing circuit defined by a cassette of the flow set of FIG. 2, which can be programmed to perform a variety of different biological fluid processing procedures in association with the device shown in FIG. 1;

FIG. 4 is a perspective view of an exemplary blood processing system; and

FIG. 5 is a perspective view of an exemplary disposable fluid flow circuit for use in the system of FIG. 4.

### DETAILED DESCRIPTION OF THE ILLUSTRATED EMBODIMENTS

The embodiments disclosed herein are for the purpose of providing an exemplary description of the present subject matter. They are, however, only exemplary and not exclusive, and the present subject matter may be embodied in various forms. Therefore, specific details disclosed herein are not to be interpreted as limiting the subject matter as defined in the accompanying claims.

The current disclosure includes devices or hardware of blood processing systems which utilize holders for maintaining the product containers in an upright position for the duration of any performed blood processing procedure.

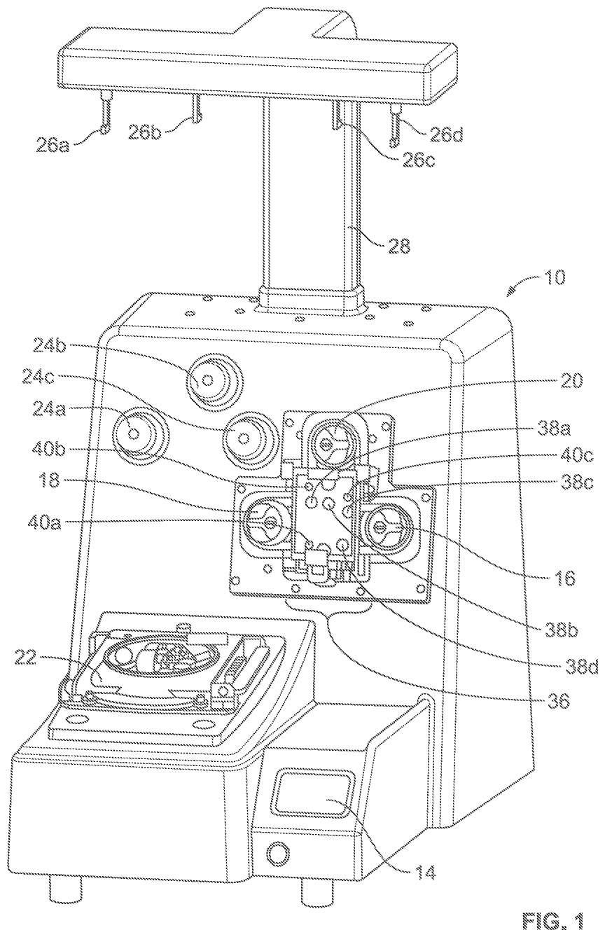

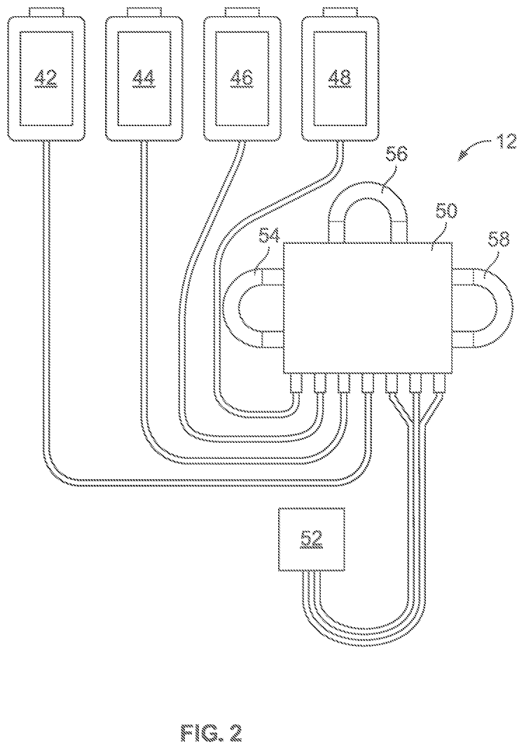

FIG. 1 depicts a reusable or durable hardware component or processing device of a configurable, automated blood processing system or blood component manufacturing system, generally designated 10, while FIG. 2 depicts a disposable or single use fluid flow circuit, generally designated 12, to be used in combination with the processing device 10 for processing collected whole blood. The illustrated processing device 10 includes associated pumps, valves, sensors, displays, and other apparatus for configuring and controlling flow of fluid through the fluid flow circuit 12, described in greater detail below. The blood processing system may be directed by a controller integral with the processing device 10 that includes a programmable microprocessor to automatically control the operation of the pumps, valves, sensors, etc. The processing device 10 may also include wireless communication capabilities to enable the transfer of data from the processing device 10 to the quality management systems of the operator.

More specifically, the illustrated processing device 10 includes a user input and output touchscreen 14, a pump station or system including a first pump 16 (for pumping, e.g., whole blood), a second pump 18 (for pumping, e.g., plasma) and a third pump 20 (for pumping, e.g., additive solution), a centrifuge mounting station and drive unit 22 (which may be referred to herein as a “centrifuge”), and clamps 24a-c. The touchscreen 14 enables user interaction with the processing device 10, as well as the monitoring of procedure parameters, such as flow rates, container weights, pressures, etc. The pumps 16, 18, and 20 (collectively referred to herein as being part of a “pump system” of the processing device 10) are illustrated as peristaltic pumps capable of receiving tubing or conduits and moving fluid at various rates through the associated conduit dependent upon the procedure being performed. An exemplary centrifuge mounting station/drive unit is seen in U.S. Pat. No. 8,075,468 (with reference to FIGS. 26-28), which is hereby incorporated herein by reference. The clamps 24a-c (collectively referred to herein as being part of the “valve system” of the processing device 10) are capable of opening and closing fluid paths through the tubing or conduits and may incorporate RF sealers in order to complete a heat seal of the tubing or conduit placed in the clamp to seal the tubing or conduit leading to a product container upon completion of a procedure.

The processing device 10 also includes hangers 26a-d (which may each be associated with a weight scale) for suspending the various containers of the disposable fluid circuit 12. The hangers 26a-d ensure that the storage bags hang in an upright position for the duration of a procedure. Specifically, the port of the containers are at the top of the bag, near the hook of the hanger. The hangers 26a-d are preferably mounted to a support 28, which is vertically translatable to improve the transportability of the processing device 10.

The face of the processing device 10 includes a nesting module 36 for seating a flow control cassette 50 (FIG. 2) of the fluid flow circuit 12 (described in greater detail below). The cassette nesting module 36 is configured to receive various disposable cassette designs so that the system may be used to perform different types of procedures. Embedded within the illustrated cassette nesting module 36 are four valves 38a-d (collectively referred to herein as being part of the “valve system” of the processing device 10) for opening and closing fluid flow paths within the flow control cassette 50, and three pressure sensors 40a-c capable of measuring the pressure at various locations of the fluid flow circuit 12.

With reference to FIG. 2, the illustrated fluid flow circuit 12 includes a plurality of containers 42, 44, 46, and 48, with a flow control cassette 50 and a processing/separation chamber 52 that is configured to be received in the centrifuge 22, all of which are interconnected by conduits or tubing segments, so as to permit continuous flow centrifugation. The flow control cassette 50 routes the fluid flow through three tubing loops 54, 56, 58, with each loop being positioned to engage a particular one of the pumps 16, 18, 20. The conduits or tubing may extend through the cassette 50, or the cassette 50 may have pre-formed fluid flow paths that direct the fluid flow.

The containers 42, 44, 46, and 48 may be configured to hold a variety of different biological and non-biological fluids, depending on the nature of the procedure to be executed. The fluid flow circuit 12 may also be provided with a different number of containers without departing from the scope of the present disclosure. In one exemplary embodiment, container 42 of the fluid flow circuit 12 shown in FIG. 2 may be pre-filled with additive solution such as Adsol, container 44 may be filled with whole blood and connected to the fluid flow circuit 12 at the time of use, container 46 may be an empty container for the receipt of red blood cells separated from the whole blood, and container 48 may be an empty container for the receipt of plasma separated from the whole blood. While FIG. 2 shows a whole blood container 44 (configured as a blood pack unit, for example) as a blood source, it is within the scope of the present disclosure for the blood source to be a living donor.

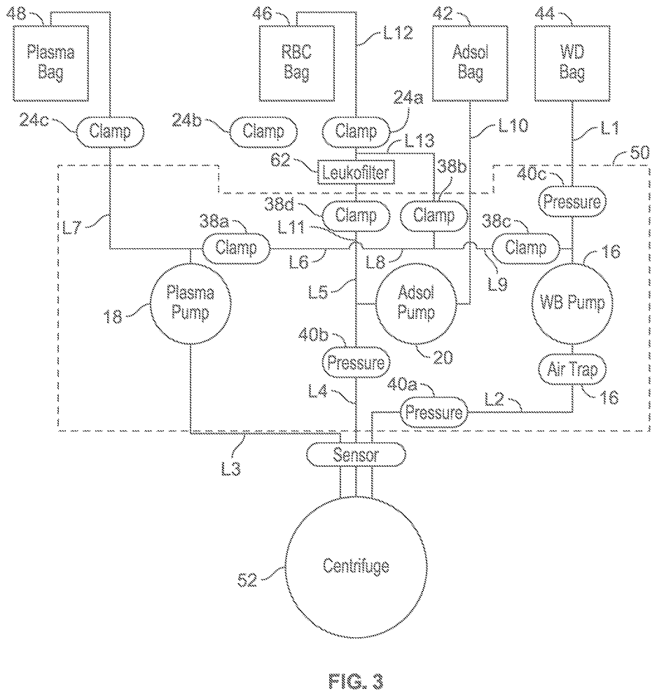

The fluid flow circuit 12 may include a variety of other features and components without departing from the scope of the present disclosure. For example, FIG. 3 illustrates a flow control cassette 50 (represented in FIG. 3 by a dashed line) including an air trap 60 (through which a fluid to be processed is flowed prior to entering the separation chamber 52). FIG. 3 shows a leukoreduction filter 62 (through which the red blood cells are flowed prior to entering the red blood cell collection container 46) positioned externally of the cassette 50. While FIG. 3 illustrates pumps, clamps, and sensors within the flow control cassette 50, it should be understood that such components are incorporated into the processing device 10 (as described above), with the flow control cassette 50 defining formations configured to interact with the components of the processing device 10 (e.g., tubing loops configured to associate with the pumps, valve chambers configured to associate with the clamps, and sensing chambers configured to associate with the sensors).

The various components of the set 12 are connected by flexible tubing to ports of the flow control cassette 50. Various tubing lines are labeled L1-L13. The cassette 50 provides a centralized, programmable, integrated platform for all the pumping and valving functions required for a given biological fluid processing procedure. In use, the cassette 50 is mounted onto the cassette nesting module 36 of the device 10. The clamps of the module 36 apply positive and negative pneumatic pressure to associated valve chambers of the cassette 50 to control and direct liquid flow therethrough, while the pumps act upon tubing loops extending from the cassette 50 to convey fluid into and out of the cassette 50. The operation of the pumps and valves to control fluid flow through the cassette 50 can be understood with reference to U.S. Patent Application Publication No. 2009/0215602, which is hereby incorporated herein by reference.

In keeping with the disclosure, the controller of the processing device 10 is programmed to automatically operate the system to perform one or more standard blood processing procedures selected by an operator by input to the touchscreen 14, and configured to be further programmed by the operator to perform additional blood processing procedures. The controller may be pre-programmed to substantially automate a wide variety of procedures, including, but not limited to: red blood cell and plasma production from a single unit of whole blood (as will be described in greater detail herein), buffy coat pooling, buffy coat separation into a platelet product (as described in U.S. Patent Application Publication No. 2018/0078582, which is hereby incorporated herein by reference), glycerol addition to red blood cells, red blood cell washing, platelet washing, and cryoprecipitate pooling and separation.

During such procedures, the device may execute a number of processing stages including, but not limited to a blood prime stage, an establish separation stage, a collection stage, a red blood cell recovery” stage, buffy coat harvest stage, an additive solution flush” stage, an “air evacuation” stage. Upon completion of the air evacuation stage, any of a number of post-processing stages may be executed. For example, a “sealing” stage in which all of the clamps and valves are closed, and all of the pumps are deactivated. In another embodiment, the fluid flow circuit 12 may be removed from the processing device 10, with lines being sealed (and optionally severed) using a dedicated sealing device.

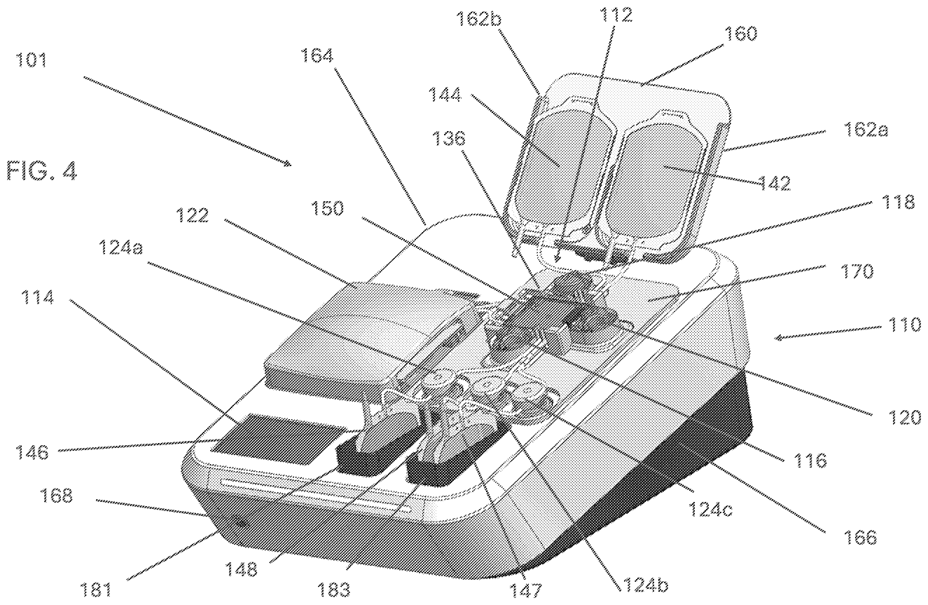

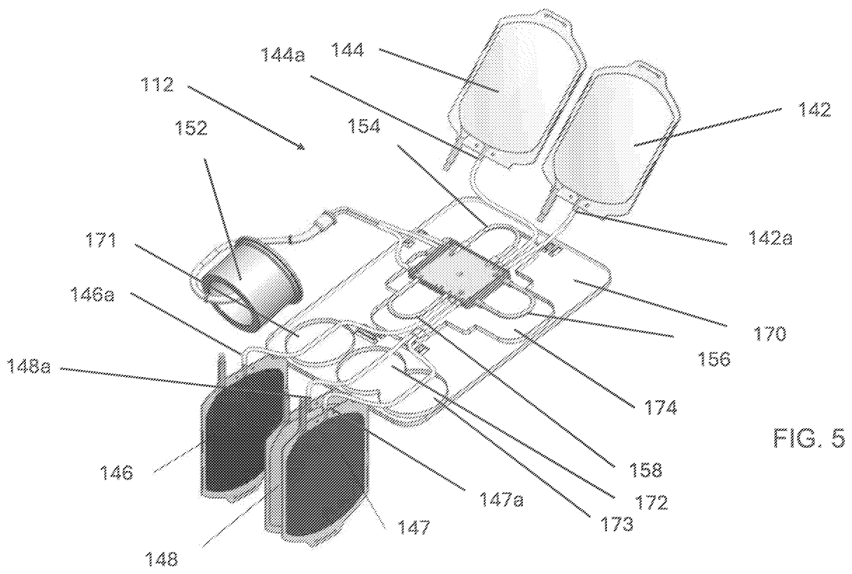

An additional embodiment of a system 101 including a device 110 and fluid flow circuit 112 is shown in FIGS. 4 and 5. For the device and fluid flow circuits of FIGS. 4 and 5, the components of the device and the fluid flow circuit of FIGS. 4 and 5 corresponding to above-described components of the device and fluid flow circuit of FIGS. 1 and 2 will be identified using corresponding reference numbers (i.e. touchscreen 14 of the embodiment of FIG. 1 corresponds to touchscreen 114 of FIG. 4), while new or differently configured components will be identified using new reference numbers. The devices and fluid circuits can run similar or even identical procedures, the principal difference being the configuration of the device, in that device 110 does not include the hangers featured in device 10, but rather alternate collection container holding devices. The collection container holding devices also enable the collection containers to be upright during the entire length of the procedure, or arranged so that the port is at the top of each collection container.

More specifically, the illustrated processing device 110 includes a user input and output touchscreen 114, a pump station or system including a first pump 116 (for pumping, e.g., whole blood), a second pump 118 (for pumping, e.g., plasma) and a third pump 120 (for pumping, e.g., additive solution), a centrifuge mounting station and drive unit 122 (which may be referred to herein as a “centrifuge”), and clamps 124a-c. The touchscreen 114 enables user interaction with the processing device 110, as well as the monitoring of procedure parameters, such as flow rates, container weights, pressures, etc. The pumps 116, 118, and 120 (collectively referred to herein as being part of a “pump system” of the processing device 110) are illustrated as peristaltic pumps capable of receiving tubing or conduits and moving fluid at various rates through the associated conduit dependent upon the procedure being performed. The clamps 124a-c (collectively referred to herein as being part of the “valve system” of the processing device 110) are capable of opening and closing fluid paths through the tubing or conduits and may incorporate RF sealers in order to complete a heat seal of the tubing or conduit placed in the clamp to seal the tubing or conduit leading to a product container upon completion of a procedure.

The device 110 includes a base 166 with a top 164. The top 164 may be slanted upward. The collection container holding devices or cups 181,183 may be molded into or separate pieces removable from the base 166. Although two separate cups are shown, one larger cup may be used, or additional cups may also be utilized. The top 164 includes openings for the collection container holding devices or cups 181, 183.

The cups may be rigid and circular or rectangular in shape. The cups are deep enough to hold a normal blood component container so that at least 75% of the collection container is within the cup or holder. The cups or holders 181, 183 may be the length, width, and depth of at least one collection container. In one embodiment the interior of each of the cups has a length of at least 5 inches. In one embodiment, the interior of each of the cups has a width of at least 2 inches. In one embodiment, the interior of each of the cups has a height of at least 5 inches. In one embodiment, the dimensions of each of the cups are 5.6 inches×2.25 inches×5.1 inches. Each cup or holder 181, 183 may be sized to accommodate a standard 600 mL collection container. Each cup or holder may be sized larger to accommodate larger collection bags.

The cups may include a retaining mechanism to further hold the collection containers in place. The retaining mechanisms may include a clamp or lever or any other known retaining mechanism.

The cups 181,183 are associated with load cells to determine the weights of the product bags. Upon separation completion, all air is removed from the top of the bags using the peristaltic pumps on the device. The pumps remove air from the bag until the clamps containing optical sensors associated with each product line detector fluid indicating all air has been removed from the bag.

The cups 181, 183 are configured to orient the collection containers upright, or so that the port (146a, 147a, 148a) of each collection container is at the top of the container. The collections containers can easily be burped or have air removed from the top of the container.

The device 110 includes a projection 160 for placing at least one blood or blood component container 144 and or additive container 142 during a blood processing procedure. The ports 142a and 144a can be located at the bottom of the bag for these containers. The projection 160 includes side bars 162a and 162b and bottom bar 163 for supporting the blood container(s) and/or additive container(s) during the procedure.

The top 164 of the processing device 110 includes a nesting module 136 for seating a flow control cassette 150 (FIG. 5) of the fluid flow circuit 112 (described in greater detail below). The cassette nesting module 136 is configured to receive various disposable cassette designs so that the system may be used to perform different types of procedures. Embedded within the illustrated cassette nesting module 136 are four valves (collectively referred to herein as being part of the “valve system” of the processing device 110) for opening and closing fluid flow paths within the flow control cassette 150, and three pressure sensors capable of measuring the pressure at various locations of the fluid flow circuit 112. The processing device 110 may also include a power on/off button 168.

With reference to FIG. 4, the illustrated fluid flow circuit 112 includes a plurality of containers 142, 144, 146, 147, and 148, with a flow control cassette 150 and a processing/separation chamber 152 shown in FIG. 5 that is configured to be received in the centrifuge mounting station 122, all of which are interconnected by conduits or tubing segments, so as to permit continuous flow centrifugation. The flow control cassette 150 routes the fluid flow through three tubing loops 154, 156, 158, with each loop being positioned to engage a particular one of the pumps 116, 118,120. The conduits or tubing may extend through the cassette 150, or the cassette 150 may have pre-formed fluid flow paths that direct the fluid flow.

The fluid flow circuit 112 additionally may include a support or tray 170 associated with the fluid flow circuit 112. The support is inserted on top of the cover 164. The support 170 includes cut-out portions for the clamps 124a, 124b, and 125c and the cassette 150. The support 170 may also include clamp cut-out portions 171, 172, 173 and a cassette cut-out portion 174. The support or tray 170 may come pre-packaged with the fluid flow circuit 112. The tray 170 enables a user to carry the disposable fluid flow circuit 170 to a device and load the fluid flow circuit 170 so that the disposable components are aligned with their hardware components. For example, cut-outs 171, 172, and 173 enable the tray/support 170 to be placed over the clamps 124a, 124b, and 124c to place the disposable tubing next to the clamps 124a, 124b, 124c for easier loading of the clamps. The tray/support 170 can be similarly configured to the tray described in U.S. patent application Ser. No. 18/420,050, filed Jan. 23, 2024.

In the fluid flow circuit 112 shown in FIG. 5, container 142 may be pre-filled with additive solution, container 144 may be filled with whole blood and connected to the fluid flow circuit 112 at the time of use, container 146 may be an empty container for the receipt of red blood cells separated from the whole blood, container 147 may be an empty container for the receipt of buffy coat separated from the whole blood, and container 148 may be an empty container for the receipt of plasma separated from the whole blood. While FIG. 5 shows a whole blood container 144 (configured as a blood pack unit, for example) as a blood source, it is within the scope of the present disclosure for the blood source to be a living donor, as will be described in greater detail herein. The fluid flow circuit may optionally include an air trap and/or a leukoreduction filter.

The controller of the processing device 110 is programmed to automatically operate the system to perform one or more standard blood processing procedures selected by an operator by input to the touchscreen 114, and configured to be further programmed by the operator to perform additional blood processing procedures. The controller may be pre-programmed to substantially automate a wide variety of procedures, including, but not limited to: red blood cell and plasma production from a single unit of whole blood (as will be described in greater detail herein), buffy coat pooling, buffy coat separation into a platelet product (as described in U.S. Patent Application Publication No. 2018/0078582, which is hereby incorporated herein by reference), glycerol addition to red blood cells, red blood cell washing, platelet washing, and cryoprecipitate pooling and separation.

During such procedures, the device may execute a number of processing stages including, but not limited to a blood prime stage, an establish separation stage, a collection stage, a red blood cell recovery” stage, buffy coat harvest stage, an additive solution flush” stage, an “air evacuation” stage. Upon completion of the air evacuation stage, any of a number of post-processing stages may be executed. For example, a “sealing” stage in which all of the clamps and valves are closed and all of the pumps are deactivated. In another embodiment, the fluid flow circuit 112 may be removed from the processing device 110, with lines being sealed (and optionally severed) using a dedicated sealing device.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

### Aspects

Aspect 1. A blood processing system, comprising: a reusable processing device including a pump system, a valve system, a centrifuge, holders for collection containers, and a controller; and a disposable fluid flow circuit including a plurality of collection containers, a processing chamber received by the centrifuge, and a plurality of conduits fluidly connecting the components of the fluid flow circuit, wherein the controller is configured to command the pump system and the valve system to cooperate to convey whole blood from a blood source to the processing chamber and execute at least one blood processing procedure; and wherein the holders are configured so that the collection containers remain upright during the duration of the at least one blood processing procedure.

Aspect 2. The system of claim 1, wherein the device further includes a platform for holding additional containers of the fluid flow circuit.

Aspect 3. The system of any of the preceding claims, wherein the device further includes a base and a top.

Aspect 4. The system of claim 3, wherein the fluid flow circuit is inserted into and on top of the base of the device.

Aspect 5. The system of any of the preceding claims, wherein the holders are cups formed in the base of the device.

Aspect 6. The system of claim 5, wherein the holders include at least two cups.

Aspect 7. The system of any of the preceding claims, further include a projection for holding at least one blood container.

Aspect 8. The system of claim 7, wherein the projection is configured to hold at least one additive container.

Aspect 9. The system of any of the preceding claims, wherein the collection containers include at least one of a red blood cell collection container, a buffy coat collection container, and a plasma collection container.

Aspect 10. The system of claim 1, wherein the holders are hooks.

Aspect 11. A method of separating blood, using the system of any one of claims 1-10.

1. A blood processing system comprising:
a reusable processing device including:
a pump system;
a valve system;
a centrifuge;
holders for collection containers;
a controller; and
a disposable fluid flow circuit including:
a plurality of collection containers;
a processing chamber received by the centrifuge; and
a plurality of conduits fluidly connecting the components of the fluid flow circuit, wherein the controller is configured to command the pump system and the valve system to cooperate to convey whole blood from a blood source to the processing chamber and execute at least one blood processing procedure; and
wherein the holders are configured so that the collection containers remain upright during the duration of the at least one blood processing procedure.

2. The system of claim 1, wherein the device further includes a platform for holding additional containers of the fluid flow circuit.

3. The system of any of the preceding claims, wherein the device further includes a base and a top.

4. The system of claim 3, wherein the fluid flow circuit is inserted into and on top of the top of the device.

5. The system of any of the preceding claims, wherein the holders are cups formed in the base of the device.

6. The system of claim 5, wherein the holders include at least two cups.

7. The system of any of the preceding claims, further include a projection for holding at least one blood container.

8. The system of claim 7, wherein the projection is configured to hold at least one additive container.

9. The system of any of the preceding claims, wherein the collection containers include at least one of a red blood cell collection container, a buffy coat collection container, and a plasma collection container.

10. The system of claim 1, wherein the holders are hooks.

11. A method of separating blood, using the system of any one of claims 1-10.
